(19) United States
(12) Patent Application Publication
(10) Pub. No.: US 2023/0311294 A1
(43) Pub. Date: Oct. 5, 2023

(54) Handheld Implement

(71) Applicants: Lisa Danielle Wisnewski, Tujunga, CA (US); Michael DeWit Kindt, Tujunga, CA (US)

(72) Inventors: Lisa Danielle Wisnewski, Tujunga, CA (US); Michael DeWit Kindt, Tujunga, CA (US)

(21) Appl. No.: 18/128,199

(22) Filed: Mar. 29, 2023

Related U.S. Application Data
(60) Provisional application No. 63/324,640, filed on Mar. 29, 2022.

(51) Int. Cl. B25G 1/10; B25F 5/02; A45D 34/04; A46B 5/02; A24F 47/00; B43K 23/00

(52) U.S. Cl. CPC B25G 1/102; A24F 47/00; A45D 34/042; A46B 5/02; B25F 5/02; B43K 23/00; A61B 2017/00424

(57) ABSTRACT

A handheld implement used in precision work requiring a user to grip the implement handle close to the tool head or tip, used in various manual activities. The implement includes a generally cylindrical handle that has a flat surface on one side formed by cutting away a segment of the cylinder down part of its length by a plane that intersects at an angle of a few degrees off the parallel of the handle’s axis, such that the handle tapers into a tail section. The resulting flat area can resemble a partially truncated or full ellipse. The generally rounded leading end can engage the thumb and fingertips of the user, while the tail’s flat area can rest generally along the side of the index finger. The handle’s tail section can have enough weight to keep the flat area flush against a table surface, elevating the tool head and preventing rolling.

### PRIORITY CLAIMS AND CROSS-REFERENCE TO RELATED APPLICATIONS

This application is related to and claims domestic priority benefits under 35 USC §119(e) from U.S. Provisional Pat. Application Ser. No. 63/324,640 filed on Mar. 29, 2022, the entire contents of which are expressly incorporated herein by reference.

### FIELD

The present disclosure relates generally to a handheld implement which is used in manual activities. Examples of such implements include cosmetics applicators, cosmetic brushes, paint brushes, pens, pencils, scalpels, dental tools, surgical tools, microblades, and various other precision instruments gripped in the hand of a user.

### BACKGROUND

Handheld implements are used for precision work that benefits from a user gripping the implement handle close to the tool head or tip. A user pinches the handle between several fingers. Cosmetic applicators are one such example in which the user attempts to create fine details on small areas of the face. The placement of the fingers close to the tool head allows fine manipulation of the implement during this work. Other precision implements like paint brushes, pencils and dental tools also perform with similar advantages of this finger placement on the handle close to the tool head.

Tool heads in some implements may have asymmetries, particularly radial asymmetries, that cause the tool to cut, write, or paint in different ways depending on the orientation of the tool head with respect to the surface.

In many applications, handheld implements are stored vertically in a container with the tool head oriented upwards. This vertical orientation is often used to provide stability and minimize contamination of the implement. Some of these delicate handheld implements are laid flat on trays.

### SUMMARY

This disclosure describes a handheld implement designed to provide a tactile cue for orienting the hand and grip in relation to an asymmetric tool head or to material applied to one section of the tool head. The implement features a user-friendly grip area and a non-rolling design to prevent it from moving around when placed on a flat surface. In addition, the handheld implement can elevate the tool head when placed on a flat surface, reducing the risk of contamination from surface contact. This versatile and hygienic handheld implement can be used with a variety of tool heads, including cosmetics, painting, writing, cutting, vape devices, handheld electronic devices, and electronic input tools. The handle is generally cylindrical in shape with a planar surface in the shape of a full or partially truncated ellipse. The planar surface is formed by cutting away a segment of the handle at an angle of a few degrees off the parallel of the handle’s axis, located towards the midsection of the handle and tapering towards the tail section. The handle can be made of various materials, have a textured or non-slip surface, and include extra weight in the tail section. The tool head may be detachable or interchangeable with other tool heads.

### BRIEF DESCRIPTION OF DRAWINGS

FIG. 1 shows a side view of a table surface having thereon an example handheld implement;

FIG. 2 shows a perspective view of the handheld implement of FIG. 1;

FIG. 3 shows another perspective view of the handheld implement of FIG. 1;

FIG. 4 shows a perspective view of a hand of a user holding the handheld implement of FIG. 1; and

FIG. 5 shows a perspective view of a hand of a user holding the handheld implement of FIG. 1.

### DETAILED DESCRIPTION

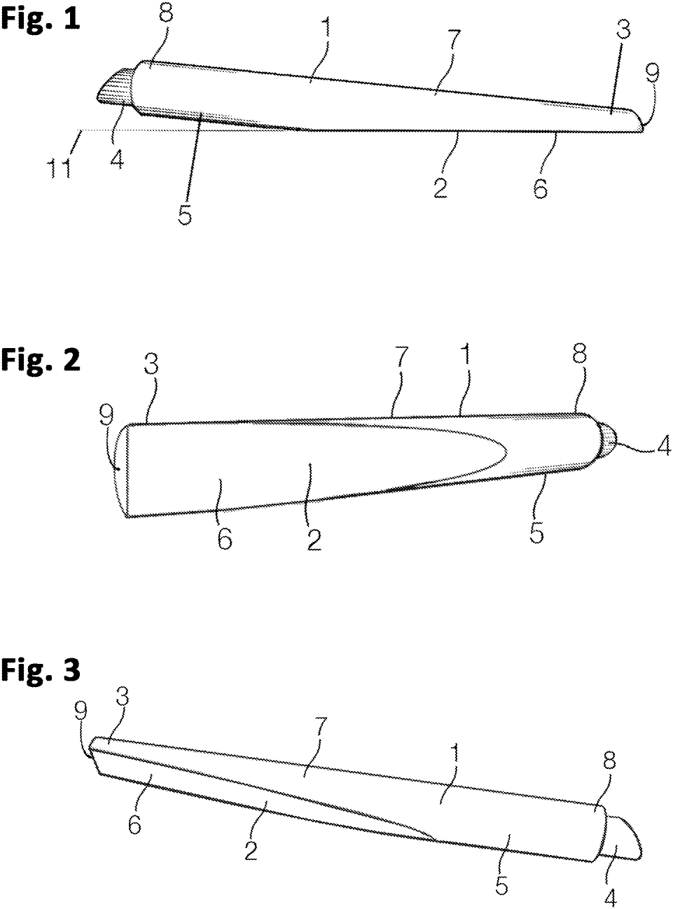

In the following illustrations (FIGS. 1 to 5), corresponding components are identified by the same reference numbers.

In the illustrated embodiment, it can be seen in FIGS. 1 through 4 that a leading end 8 of a handle 1 of the handheld implement is attached to a tool head 4, in this embodiment a cosmetic brush head made of natural bristles or plastic fibers. However, this tool head 4 can be any of various precision tools controlled by fingers positioned on a leading end area 5 of the handle 1 close to the tool head 4, including writing implements such as calligraphy pens and pencils, medical tools such as scalpels, artist tools such as paint brushes, and other cosmetic tools such as foam tipped applicators and lipsticks.

Referring to FIGS. 1 through 3, a handle 1 that is generally cylindrical in shape is provided with a planar surface 2 by cutting away a segment down approximately half its length by a plane. The plane intersects at an angle of a few degrees off the parallel of the handle’s axis, such that the handle 1 tapers in a tail section 7. In this embodiment, the planar surface 2 formed is in the general shape of a partially truncated ellipse with a shortened tail tip 3 forming a minor segment of a circle defined by an arc and a chord 9. In some embodiments the ellipse is not necessarily truncated, and can be a full ellipse extending completely along the handle 1 to a rounded tail.

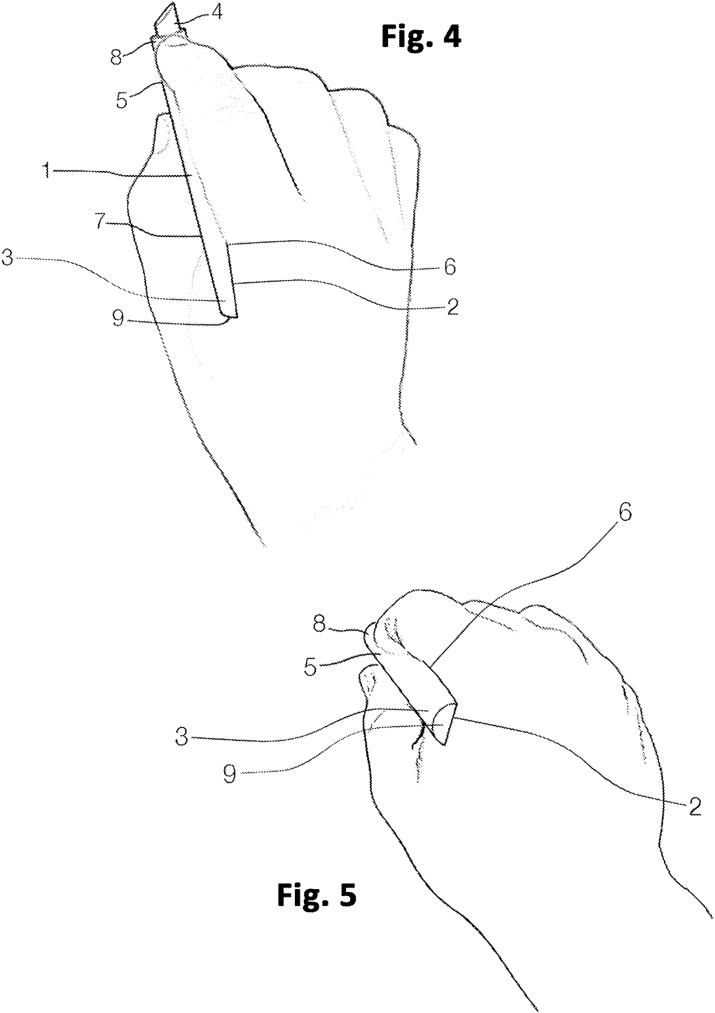

In this embodiment, FIGS. 4 and 5 show one possible grip configuration of the implement in the user’s hand. The handle 1 of the handheld implement can rest substantially along the side of the index finger, with a broad contact area 6 of the planar surface 2 touching the hand close to where the user’s thumb and index finger connect. In another example of a grip based upon user preference, the planar surface 2 can naturally come to rest towards the knuckle at the base of the user’s index finger. With the planar surface 2 coming to rest against this area of the hand, the implement is stabilized and oriented in a specific alignment. The planar surface 2 of the handle 1 thus provides a tactile cue for orienting the implement in the hand.

Using only the tactile cue of the planar surface 2 of the handle 1, the user can hold the implement in a fixed orientation without having to study the tool visually. In some embodiments having tool heads that are radially symmetrical, this fixed orientation of the implement can enable the user to keep track of and control application of product that is placed on the tool head 4 at a specific angle. For instance, a foam cosmetic brush can have powder placed asymmetrically on one small section of the foam head.

In the illustrated embodiment, the tool head 4 is generally round but cut at an angle such that the tip is off-center. Lacking symmetry about its axis, the tool head 4 is radially asymmetric. This tool head 4 is specifically oriented in relation to the planar surface 2 of the handle 1. Thus, the broad contact area 6 of the planar surface 2 that rests against the user’s hand can gently constrain the brush position and can help to align a radially asymmetric tool head 4. This tactile cue can help especially when the tool head 4 is positioned in close proximity to the working surface and the user’s hand may obscure their line of sight, making it difficult to accurately determine the orientation of the tool head 4 or product applied to the tool head 4.

In this embodiment, the center of gravity of the handle 1 is located within the handle’s tail section 7 such that the planar surface 2 remains flush against a table surface 11. The planar surface 2 extends far enough along the handle 1 from the tip of the tail 3 towards the leading end 8 such that the center of gravity, when the tool is placed on a flat surface, is far enough away from the head 4. Thus, the handle 1 rests on the table surface 11 with the tool head 4 elevated. In some embodiments, extra weight in the tail section 7 of the handheld implement also changes the center of gravity to prevent the implement from falling toward the tool head 4 on the table surface 11. The stability of this planar surface 2 also prevents rolling of the handle 1 on the table surface 11. The stability of the implement and elevation of the implement tool head 4 is particularly helpful for preventing contact between the table surface 11 and the tool head 4, improving cleanliness and reducing contamination concerns. Stabilizing the handheld implement on the table surface 11 can make it unnecessary to store the handheld implement in a vertical container, where contamination from other implements within the same container can occur, and may also result in the user contaminating the tool head 4 when attempting to remove the implement. Vertical placement of tools with wet tool heads can also result in contamination of the handle, as gravity pulls the liquid or moisture downwards. Furthermore, in a vertical container, if the tool head 4 is sharp, such as in the case of a scalpel or dental explorer, there is a risk of injury to the user.

In the illustrated embodiment, because the planar surface 2 is limited mainly to the tail section 7 of the handle, the leading end area 5 can be generally cylindrical. In use, the generally cylindrical regions of the leading end area 5 as well as the rounded part of the tail section 7 give the user the flexibility of choosing a variety of natural configurations of finger placement along the handle 1. FIGS. 4 and 5 show one placement of index finger, middle finger and thumb in this leading end area 5. In another configuration the thumb could move further down toward the tail section 7, resting on the rounded part of the tail section 7. In yet another configuration the middle finger would extend past the leading end 8, the implement resting near the top knuckle of the middle finger, while the index finger and thumb would be placed close together further toward the tail section 7 while still in the rounded leading end area 5 for very controlled precision use. Thus, the handheld implement grip can offer an unrestricted experience for the user. In combination with the planar surface 2 helping to orient the handheld implement, this embodiment orients the tool head 4 without forcing the user to use a specific grip and allows subtle grip changes for detailed work like makeup application.

The dimensions of the implement can vary to align with the aforementioned criteria. In the illustrated embodiment, the planar surface 2 is formed by cutting away a segment down approximately half its length by a plane that intersects at an angle of approximately 5 degrees off the parallel of the handle’s axis, such that the handle 1 tapers in a tail section 7. The angle of the plane that intersects with the handle 1 can vary in different embodiments, ranging from 1 to 20 degrees off the parallel of the handle’s axis. The handheld implement can be manufactured in different sizes to accommodate various hand sizes and tool heads.

In some embodiments the tool head 4 can be detachable, allowing for easy replacement or maintenance. In other embodiments, the tool head 4 can be interchangeable with other tool heads, providing even greater flexibility in its use. The handle 1 of the implement can include a textured surface and a non-slip material that enhances grip. The handle 1 may have a generally oval cross-sectional shape in certain embodiments.

The examples described are not limited to specific materials nor are they limited to specific tool heads. Examples include tool heads that are sponge, rubber, plastic, natural bristles, ink pens, metal needles, microblades, soldering tools, surgical tools, dental tools, vape pens, styluses, and medical devices. Other physical characteristics are not limited to the example embodiment, but can include various styles, shapes, surfaces and textures.

What is claimed is:

1. A handheld implement comprising:
a handle that is generally cylindrical in shape having a leading end and a tail section;
a planar surface in the general shape of a full ellipse, formed on the handle by cutting away a segment of the handle down part of its length, at an angle of a few degrees off the parallel of its axis, the planar surface located from the tail section towards a midsection of the handle, so as to taper the tail section; and
a tool head attached to the leading end of the handle;.

2. The handheld implement of claim 1, wherein the planar surface of the handle is oriented in relation to a radially asymmetric tool head;.

3. The handheld implement of claim 1, wherein the planar surface extends sufficiently along the handle from the tail section towards the leading end such that, when the handheld implement is placed with the planar surface downward on a flat surface, the center of gravity is located within the tail section above the planar surface.

4. The handheld implement of claim 1, wherein the planar surface is in the general shape of a partially truncated ellipse, such that the end of the tail section has a tip in the shape of a minor segment of a circle defined by an arc and a chord of the handle.

5. The handheld implement of claim 1, further comprising a tool head selected from the group consisting of cosmetic tools, including but not limited to brushes, sponges, and applicators.

6. The handheld implement of claim 1, further comprising a tool head selected from the group consisting of painting tools, including but not limited to brushes, silicone squeegees, and rollers.

7. The handheld implement of claim 1, further comprising a tool head selected from the group consisting of writing tools, including but not limited to pens and pencils.

8. The handheld implement of claim 1, further comprising a tool head selected from the group consisting of cutting tools, including but not limited to scalpels, dental tools, surgical tools, and microblades.

9. The handheld implement of claim 1, further comprising a vape device as the tool head.

10. The handheld implement of claim 1, further comprising a tool head selected from the group consisting of handheld electronic devices and electronic input tools.

11. The handheld implement of claim 1, wherein the planar surface on one side of the handle is formed by cutting away a segment of the cylinder down a plane that intersects at an angle ranging from 5 to 20 degrees off the parallel of the handle’s axis.

12. The handheld implement of claim 1, wherein the planar surface on one side of the handle is formed by cutting away a segment of the cylinder down a plane that intersects at an angle ranging from 1 to 5 degrees off the parallel of the handle’s axis.

13. The handheld implement of claim 1, wherein the handle is made of a material selected from the group consisting of metal, plastic, wood, and composite materials.

14. The handheld implement of claim 1, wherein the tool head is detachable.

15. The handheld implement of claim 1, wherein the tool head is interchangeable with other tool heads.

16. The handheld implement of claim 1, wherein the handle has a textured surface.

17. The handheld implement of claim 1, wherein the handle is made of a non-slip material.

18. The handheld implement of claim 1, wherein the handle includes extra weight in the tail section.

19. The handheld implement of claim 1, wherein a generally rounded area of the handle has an oval cross-section.
